(19) United States
(12) Patent Application Publication
(10) Pub. No.: US 2008/0305144 A1
(43) Pub. Date: Dec. 11, 2008

(54) High Strength Devices and Composites

(76) Inventors: Malcolm NMI Brown, York (GB); Michael Andrew Hall, Middlesbrough (GB); John NMI Rose, York (GB); Alan NMI Bull, York (GB); David F. Farrar, York (GB)

Correspondence Address: CHIEF PATENT COUNSEL;SMITH & NEPHEW, INC., 1450 BROOKS ROAD, MEMPHIS, TN, 38116, US

(21) Appl. No.: 12/064,156

(22) PCT Filed: Aug. 16, 2006
(86) PCT No.: PCT/GB2006/003049

§ 371 Date: Jul. 21, 2008

(30) Foreign Application Priority Data
Aug. 18, 2005 (GB) 0516943.8
Nov. 16, 2005 (GB) 0523318.4

(51) Int. Cl. A61L 27/14; A61F 2/00; A61L 17/12

(52) U.S. Cl. 424/423; 523/113; 264/239

(57) ABSTRACT

An oriented implantable, biodegradable device is disclosed. The oriented implantable, biodegradable device is formed from a homogeneous polymer blend comprising a polylactic acid in admixture, in an amount of not more than 10% by weight of the polymer blend, with an additive which both plasticises polymer draw and is a degradation accelerant. The polymer comprised within the blend may be a uniaxial, biaxial or triaxial orientation. Also disclosed is a composite thereof, processes for the preparation thereof, and The implantable biodegradable device may be used as a high strength trauma fixation device suitable for implantation into the human or animal body. As examples, the high strength trauma fixation device may take the form of plates, screws, pins, rods, anchors or scaffolds.

This application claims the benefit of U.K. Provisional Application No. 0516943.8 filed Aug. 18, 2005 and U.K. Provisional Application No. 0523318.4, filed Nov. 16, 2005 both entitled “High strength fibres and composites” and the entire contents of which are hereby incorporated by reference.

### TECHNICAL FIELD OF THE INVENTION

This invention relates to biodegradable polymeric devices and composites, particularly to bioresorbable devices and composites and to artifacts made therefrom and their uses.

### BACKGROUND OF THE INVENTION

High strength trauma fixation devices (plates, screws, pins etc) are presently made of metal, typically titanium and stainless steel, however metal devices have several well known disadvantages.

Currently amorphous or semi-crystalline bioresorbable polymers such as poly (glycolic acid) (PGA) and poly (lactic acid) (PLA) are typically used to produce low load bearing devices—such as suture anchors, screws or tacs. One of the main criteria for using resorbable materials is that they carry out a mechanical function, degrade within a reasonable timeframe (for example, less than 3 yrs), and are ideally replaced by bone when used in bone sites. However, these materials are not used in high load bearing applications because they are not strong or stiff enough to resist deformation under high load.

To overcome these deficiencies, a composite approach has been applied to generate stiffer and/or stronger bioresorbable materials. Poly (L) lactic acid (P(L)LA) fibre—composites are known. Drawn P(L)LA fibres and monoliths are also known. By incorporating drawn materials, such as fibres and rods in which the polymer is oriented in the direction of drawing, the directional strength is dramatically increased. Products from these combined approaches include fibre reinforced composites, using drawn fibre and a polymer matrix, and self-reinforced materials, using extruded billets which are die drawn into self reinforced rods.

However, drawn P(L)LA fibres are reported to have longer degradation times of up to 40 years

To address this deficiency, acid accelerants have been incorporated into the P(L)LA polymer (WO 03/004071) to produce faster degrading P(L)LA blocks. However it is known that adding these accelerants to the polymer plasticises the base polymer. Whilst the incorporation of these acid accelerants does not seriously compromise the mechanical properties of moulded P(L)LA block, the incorporation of a plasticiser into high strength fibre is expected to reduce both the strength and modulus of the drawn fibres.

WO 01/46501 discloses preparing a melt blend of polyester and multicarboxylic acid having improved processability in an extruder and also having improved crystallization and absorption properties, envisaged for use in manufacturing nappies.

In U.S. Pat. No. 5,527,337 there is disclosed a biodegradable stent formed from woven lactide polymer fibres wherein, inter alia, an excipient such as citric acid or fumaric acid can be incorporated with lactide polymers during the polymer processing to improve biodegradability. However, the fibres of WO 01/46501 and U.S. Pat. No. 5,527,337 are not required to have a high load-bearing performance, but rather are intended to display elastic or absorptive properties and, therefore, would not be suitable for the presently envisaged applications.

Thus it would be desirable to produce a PLLA high strength fibre and composite with a reduced degradation time without compromising the mechanical properties of the composite.

We have now surprisingly found that high strength fibres can be produced by incorporating plasticisers in the polymer blend, such as lauric acid, a fatty acid known from WO 03/004071, to plasticise and accelerate the degradation of P(L)LA, and drawing the fibres to orient the polymer. Surprisingly the mechanical properties of drawn fibre were not compromised by the incorporation of plasticiser. We have also found that incorporating these plasticisers increased the degree of draw of the fibres during conventional hot drawing but decreased the drawing temperature.

### SUMMARY OF THE INVENTION

Thus, in accordance with the broadest aspect of the present invention there is provided an oriented implantable, biodegradable device formed from a homogeneous polymer blend comprising a poly lactic acid (PLA) in admixture, in an amount of not more than 10% by weight of the polymer blend, with an additive which plasticises polymer draw and which is a degradation accelerant wherein polymer comprised within the polymer blend is in uniaxial, biaxial or triaxial orientation.

Suitably, the additive is biocompatible. The additive may be suitable for any application and is advantageously suitable for use in medical applications. In an embodiment of the invention the additive is a carboxylic acid or precursor thereof. As an example, an acid precursor is a carboxyl containing compound and is selected from an acid anhydride, ester or other acid precursor. The acid may be a mono or poly saturated or unsaturated acid, more particularly a mono or diacid. In an embodiment of the invention the acid is a monoacid or precursor thereof. The acid is suitably a C₄₋₂₄ carboxylic acid or precursor.

Examples of suitable additives include the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-beta-hydromuconic acid, isovaleric anhydride, hexanoic anhydride, decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride and phthalic anhydride and mixtures thereof.

In a particular advantage the oriented PLA polymer of the invention is characterised by improved degradation properties, equivalent mechanical properties and enhanced draw with respect to the original drawn PLA polymer.

Reference herein to an oriented device is to a device comprised of oriented polymer as known in the art, also known as aligned polymer, wherein the polymer is in uniaxial, biaxial or triaxial alignment. As known in the art, polymers comprise discrete polymer chains which may be aligned or oriented to render the polymer in uniaxial, biaxial or triaxial alignment. Alignment or orientation is suitably conferred by further processing in suitable manner and as hereinafter defined. The oriented device of the invention is therefore distinct from polymer which has not been further processed to confer orientation, and in which polymer chains are typically in random alignment. Orientation may be determined by techniques as known in the art for example scanning electron microscopy (SEM), transmission electron microscopy (TEM), differential scanning calorimetry (DSC), X-ray, optical microscopy and the like.

We have found that a advantageous additive for use in the invention is lauric acid or benzoic acid. This may be employed as the acid per se or, if desired, as a precursor, for example as the anhydride.

In an embodiment of the present invention, the additive will not only control the rate of degradation but will delay the onset of the degradation process relative to the acid. This delay may be achieved, aptly by the use of precursors which are convertible to the acidic form of the additive. Suitable precursors are acid anhydrides which will, in an in vivo environment, hydrolyse to the corresponding acid. Example anhydrides include lauric anhydride and benzoic anhydride.

Aptly the polymer blend will contain not more than 5%, and more aptly not more than 2%, by weight of the additive and typically the blend will contain not more than 1% by weight of the additive. Example blends will contain not more than 2%, ideally not more than 1%, by weight of the blend of lauric acid or a precursor thereof.

The amount of the additive chosen will also depend upon the nature of the additive and the rate of degradation desired.

The polymeric component of the polymer blends useful for the invention essentially comprise a biodegradable PLA, including homopolymers, (block) copolymers, blends, individual or mixed isomers and the like, which may be bioresorbable, bioerodible or display any other form of degradation, for example instability to water, heat or acid, polymer. The PLA may be suitable for any application and is advantageously suitable for medical applications, for example is suitable for implantation into the human or animal body. An oriented device of the invention may be single phase (amorphous) or biphasic (semi crystalline and amorphous). Suitably blends are of miscible polymers.

Suitable biodegradable PLA's are selected from poly(lactic acid), isomers thereof including P(L)LA, P(D)LA, P(D,L)LA, blends and copolymers thereof.

A co-polymer for use in the blend of the invention may comprise more than one PLA as hereinbefore defined or may comprise other known biodegradable polymeric components copolymerised therewith, such as polyesters, including poly(lactic acid), poly(glycolic acid), copolymers of lactic and glycolic acids, copolymers of lactic and glycolic acid with poly(ethylene glycol), poly(ε-caprolactone), poly(3-hydroxybutyrate), poly(p-dioxanone), poly(propylene fumarate), poly(trimethylene carbonate) and the like. In embodiments of the invention the copolymer is a copolymer with another poly(lactic acid) or with poly glycolic acid, for example a co-polymer of poly(lactic acids) such as P(L)LA/P(D)LA copolymer, or a copolymer of poly(lactic acid) and glycolic acid (known as PLA/PGA co-polymer).

Additionally the blend may, in addition to the additive, consist of a blend of PLAs or copolymers as hereinbefore defined with other biodegradable polymeric components, for example, polyesters, for example a blend of polylactic acid or PLA/PGA co-polymer either alone or in admixture with each other.

Molecular weight of the polymeric component may be selected according to the particular polymer to be used and the intended use of the device of the invention, and therefore the required strength and modulus. In embodiments of the invention the polymeric component has number average molecular weight (Mn) in oriented form in the range in excess of 30,000 daltons or alternatively in the range 50,000 to 500,000 daltons. Molecular weight of the oriented device for higher strength applications, for example oriented fibres, may be in the range 100,000 to 400,000 daltons. Molecular weight may be determined in known manner, for example by gel permeation chromatography (GPC), viscometry or the like.

Suitably polymeric component is selected with an intrinsic viscosity (IV), in the range 1 to 10, and more particularly 2 to 5.

The oriented device may also contain fillers such as osteoconductive materials and the like and/or biological actives such as hydroxyapatite.

The oriented device of the invention may be provided in the form of fibres, drawn monoliths such as rods and the like, spun or moulded devices or may be used to produce high strength composites reinforced by component fibres, drawn monoliths, spun or moulded polymer and the like. Fibres may be continuous or chopped. Reference herein to fibres includes fibres, yarns, strands, whiskers, filaments, ribbons, tapes and the like. Drawn devices may be singly or multiply drawn.

The oriented device of the invention is characterised by properties of high strength. In embodiments of the invention the device has a tensile strength in excess of about 150 MPa up to about 2000 MPa depending on the polymer components and the form thereof. Tensile strength may be in the range from about 800 to about 2000 MPa, for example about 800 to about 1000 or about 1000 to about 2000 MPa, for fibre form devices, or in the range about 150 to about 800 MPa for drawn monoliths, spun or moulded polymer. This compares with a tensile strength of undrawn PLA fibres of the order of 70 MPa.

In a further aspect of the invention there is provided a composite comprising an oriented device as hereinbefore defined In embodiments of the invention the polymer matrix is a bioresorbable polymer, and may be selected from any bioresorbable polymer, for example a polyester, such as PLA or the like and its isomers and copolymers and blends thereof as hereinbefore defined.

In an embodiment of the invention the polymer matrix is selected from PLA, P(L)LA, P(D)LA, P(D,L)LA, PGA, polycaprolactone (PCL) and the like and (block) copolymers and blends thereof.

A matrix polymer may be formed from a homogeneous polymer blend comprising the polymer(s) in admixture, in an amount of not more than 10% by weight of the polymer blend, with an additive which plasticises polymer and which is a degradation accelerant as hereinbefore defined.

A composite of the invention may also contain fillers such as osteoconductive materials and/or biological actives such as hydroxyapatite in the matrix and/or the oriented device.

In an embodiment of the invention the composite comprises oriented device present in a known manner, for example provided as random or aligned fibres, a fabric in woven or unwoven or braided form or as a scrim, mesh, preform or prepreg. Fabrics may be mats, felts, veils, braided, knitted, punched, non-crimp, polar-, spiral- or uni-weaves, tailored fibre placement fabrics and the like. Composite may comprise continuous or chopped oriented fibres of the invention.

The oriented device may be present in any desired amount, for example in an amount of from about 1 wt % to about 70 wt % of the composite.

A composite of the invention is biodegradable and may comprise any implantable device where temporary residence only is required. Examples of such devices include suture anchors, soft tissue anchors, interference screws, tissue engineering scaffolds, maxillo-facial plates, fracture fixation plates and rods and the like.

The composite of the invention is characterised by properties of high strength. For example, the composite of the invention has tensile strength in excess of 150 MPa up to 800 MPa depending on the constituent polymer components and matrix polymer and the composite form. Tensile strength is, for example, in the range about 250 to about 550 MPa, for example about 350 to about 500 MPa comprising fibre form devices, drawn monoliths, spun or moulded devices.

In a further aspect of the invention there is provided a process for the preparation of an oriented device as hereinbefore defined comprising preparing a polymer blend comprising a polylactic acid in admixture, in an amount of not more than 10% by weight of the polymer blend, with an additive which plasticises polymer draw and which is a degradation accelerant as hereinbefore defined, and processing to orient polymer whereby polymer is in uniaxial, biaxial or triaxial orientation.

Polymer component is commercially available or may be prepared by processes as known in the art.

The polymer blends used for the present invention may be produced by known processes such as solution blending wherein the additive is blended directly into a solution of a polymeric component comprising for example, PLA in chloroform, by melt blending in melt phase, or by dry blending the solid polymer and additive materials and then solution blending the solid mixture with solvent such as chloroform. The solution blend is then dried to form a solid blend or is cast onto a surface and dried.

In an embodiment of the invention the polymer blend is cast, compression moulded or extruded into a form suitable for shaping and orienting, for example moulding or extruding as monolith such as billets or rods, or fibre or film, and oriented by any known process that induces orientation into a polymer, selected from uniaxial, biaxial or triaxial orientation as hereinbefore defined.

Casting or compression-moulding may be conducted by rendering the solid blend in melt phase for shaping into a desired form for orienting. Extrusion may be of powder or pellets as a dry blend from a hopper with extrusion via a suitable die to the desired shape.

In an embodiment of the invention orienting is by aligning melt phase polymer blend and cooling, more particularly by drawing, spinning or moulding melt phase polymer blend to orient polymer chains in the direction of draw or spin, or axis or direction of moulding, and cooling, such as drawing, for example fibre drawing produces increased strength and modulus fibre, or (hydrostatic) die drawing produces an increased strength or modulus rod or the like, spinning for example gel spinning or solution spinning produces increased strength or modulus fibre, moulding for example Shear Controlled Orientation in Injection Moulding (SCORIM) produces increased strength or modulus fibre, rod or shaped polymer, and the like. Preferably high strength oriented device may be produced by processing to orient the polymer using any of the following processes:—

By fibre drawing to produce a high strength-high modulus fast degrading polyester fibre (e.g. P(L)LA fibre);

By hydrostatic die drawing or die drawing to produce a high strength-high modulus fast degrading polyester rod (e.g. P(L)LA rod);

By solution processing such as gel spinning or solution spinning (to produce fibre at ambient temperature from solution, with subsequent solvent removal;

By SCORIM or the like to produce monoliths with orientation by shearing effect of pistons.

Drawing, spinning and moulding processes are known in the art. Drawing is undertaken, for example, by feeding the moulded film or extrudate at elevated temperature through a die and drawing the polymer, whereby the polymer chains orient in the direction of drawing, and cooling. Drawing may be conducted in two stages or passes.

In a further aspect of the invention, an oriented device of the invention may be used to prepare a polymer composite as hereinbefore defined. Composites according to the invention may be prepared by providing the oriented device in desired form and combining with matrix polymer as hereinbefore defined. Matrix polymer is suitably combined in solid, solution or melt form with the oriented device in accordance with the invention and hardened for example by moulding, compression moulding or drying. Matrix may be combined by blending, impregnation, infusion, injection or the like as known in the art. In an embodiment of the invention an additive-containing blend as hereinbefore defined may be utilized to prepare both oriented device and matrix component of a composite material which is then fabricated into a high strength biodegradable composite device as hereinbefore or hereinbelow defined.

In a further aspect of the invention there is provided the use of an oriented device or a composite thereof as hereinbefore defined as an implantable biodegradable device such as a high strength trauma fixation device suitable for implantation into the human or animal body, for example plates, screws, pins, rods, anchors or scaffolds, in particular suture anchors, soft tissue anchors, interference screws, tissue engineering scaffolds, maxillo-facial plates, fracture fixation plates and rods and the like.

### BRIEF DESCRIPTION OF THE DRAWINGS

The present invention will be illustrated in non-limiting manner by reference to the following examples and accompanying figure and drawings wherein:

FIG. 1 illustrates the degradation profile of drawn P(L)LA and drawn P(L)LA/Lauric acid (LA) fibres with time.

FIG. 2 illustrates the molecular weight (Mn) change of drawn P(L)LA and drawn P(L)LA/LA fibres with time.

### EXAMPLE 1

### P(L)LA/Lauric Acid (LA) Blend Production

The following blends were produced by solution blending:—

Polymer P(L)LA IV=3.82(213.21 g)(Purac)+lauric acid (LA) (1.07 g) (I)

Polymer P(L)LA IV=4.51(213.21 g)(Purac)+lauric acid (LA) (1.07 g) (II)

### Methods

The solids placed in each jar were thoroughly mixed by shaking the jars and split into 3 separate jars. 950 mL of CHCl₃ was added to each of the 3 jars, and the jars placed on agitating rollers for around 10 hours to dissolve the polymer.

Once the polymer was dissolved, the viscous solutions were cast by poured them out into release paper trays, in order to produce thick sheets of polymer suitable for subsequent granulation. The sheets were left to dry for 2 days in the fume cupboard before undergoing the subsequent drying procedure:

60° C. under vacuum for 6.5 h+room temperature under vacuum overnight;
80° C. under vacuum for 5 h+room temperature under vacuum over weekend; or
100° C. under vacuum for 4 h

The sheets were then granulated with the Cumberland mechanical grinder (fitted with 3 mm sieve), after having been dipped into liquid nitrogen to render them more brittle. All the granules were further dried under vacuum at 100° C. for 4 hours, and left under vacuum at room temperature for 3 days.

### EXAMPLE 2

### Fibre Production

The following methodology was used to produce both P(L)LA and P(L)LA/LA fibres

### Method

The polymer (P(L)LA-IV=3.8/Purac) or polymer blend (P(L)LA-IV 3.8/Purac/lauric acid) was extruded using a Rondol 12 mm extruder. The extruder was fitted with a general-purpose 12 mm screw with a 25:1 L/D ratio. The extruder was fitted with a 2 mm (diameter) die (coated) with a L/D ratio of 6:1. The fibre was produced using a flat temperature profile of 240° C. for all zones. A nominal 0.5 mm diameter fibre was produced (using maximum screw speed of 50 rpm) and hauled off at a rate of 16 meters per minute. The diameter of the fibre was monitored during the run using a Mitutoyo laser micrometer. The extruded fibre was sealed in a foil pouch containing a desiccant sachet and then stored in a freezer at −20° C. prior to further processing.

### EXAMPLE 3

### Drawn Fibre

The following methodologies were used to draw both P(L)LA and P(L)LA/LA fibres

Method 1: Hot shoe

Fibre drawing was carried out using a customised drawing rig. The rig consists of two sets of godets and heated plate (hot shoe). The godets were preset to rotate at different speeds. The fibre was feed from a spindle, through the 1^{st} set of godets, drawn over the hot shoe and around the 2^{nd} set of godets. The drawn fibre was finally collected on a Leesona fibre winder.

### Results

The fibres were drawn under various temperatures and speeds to produced fibres with different properties as shown in Table 1.

TABLE 1

| | | | | | | | | | |
|---|---|---|---|---|---|---|---|---|---|
| Temp 1^{st} | Speed of | Speed of | Temp 2^{nd} | Speed of | Speed of | Fibre | | Max | |
| pass | Godet 1 | Godet 2 | pass | Godet 1 | Godet 2 | Dia | Total | stress | Modulus |
| (C.) | (RPM) | (RPM) | (C.) | (RPM) | (RPM) | (mm) | draw | (MPa) | (GPa) |

(a) PLLA/LA

| | | | | | | | | | |
|---|---|---|---|---|---|---|---|---|---|
| 160 | 3 | 37 | — | — | — | 0.138 | 209.0 | 529.8 | 9.5 |
| 150 | 3 | 42 | — | — | — | 0.127 | 248.0 | 689.6 | 10.9 |
| 160 | 4 | 16 | 180 | 10 | 27 | 0.099 | 405.0 | 823.0 | 8.4 |

(b) PLLA

| | | | | | | | | | |
|---|---|---|---|---|---|---|---|---|---|
| 160 | 3 | 24 | — | — | — | 0.136 | 216 | 738 | 11.6 |
| 180 | 3 | 34 | — | — | — | 0.145 | 190.2 | 714 | 10.57 |
| 160 | 4 | 16 | 180 | 10 | 18 | 0.178 | 126.2 | 759.0 | 8.7 |

These drawn fibres were mechanically tested, using an Instron 5566 with a crosshead speed of 10 mm/min and a gauge length of 35 mm. The fibre diameter was measured using callipers and micrometers. The total draw ratio (defined as the square of initial fibre diameter/square of final fibre diameter) is given with the strength and modulus data below. Incorporating the LA increased the degree of draw of the fibres during conventional hot drawing at decreased drawing temperature.

Method 2: Zone drawn

Fibre was drawn using a batch zone drawing process. A cylindrical brass zone (outer diameter 25 mm, inner diameter 5 mm, 63 mm length) was attached to a moving plate. The temperature was controlled using a temperature probe connected to the zone and temperature controls. A clamp was fixed at a given height above the zone, 1 metre length of fibre was clamped at 1 end, threaded through the zone (using a brass rod), and the load was attached to the free end of the fibre. Fibre was then drawn under various speed, load and zone temperatures.

The samples were drawn using the following drawing conditions shown in Table 2.

TABLE 2

| | Draw | | Draw | | | | Max | |
|---|---|---|---|---|---|---|---|---|
| | Temp | Load | Temp | Load | Diameter | Total | Stress | Modulus |
| | (C.) | (g) | (C.) | (g) | (mm) | draw | (MPa) | (GPa) |
| PLLA/LA (IV 3.8) | 160 | 100 | 180 | 100 | 0.141 | 201 | 742.78 | 8.83 |
| (0.5 mm ext fibre) | 160 | 50 | 180 | 150 | 0.15 | 178 | 744.54 | 9.92 |
| (**) PLA (IV 3.8) | | | | | | | | |
| (0.35 mm ext fibre) | 160 | 50 | 180 | 100 | 0.107 | 349 | 895 | 9.98 |
| (*) PLLA/LA (IV 4.5) | 160 | 50 | 180 | 200 | 0.15 | 178 | 674.56 | 8.95 |
| (0.5 mm ext fibre) | 160 | 100 | 180 | 200 | 0.16 | 156 | 795.34 | 9.57 |
| | 160 | 100 | 180 | 300 | 0.14 | 204 | 990.09 | 9.99 |
| (**) PLLA (IV 4.5) | 180 | 150 | — | — | 0.147 | 185 | 663 | 11.57 |
| (0.35 mm ext fibre) | 160 | 100 | 180 | 170 | 0.111 | 325 | 1074 | 9.93 |

Draw Speed
(*) = 200 mm/min,
(**) = 50 mm/min

### Results

The results showed equivalent results for drawn fibres with and without LA additive.

### EXAMPLE 4

### Lauric Acid Determination

### Method

Approx. 0.1 g of each PLA/LA sample was dissolved in approx. 10 ml chloroform. Internal standard was added via glass pipette (0.9 mg/ml Hexanoic acid in acetone). Samples were left overnight to dissolve. 20-30 ml of diethyl ether was added to precipitate the polymer. An aliquot of each solution was filtered through 0.45 μm PTFE GDX Whatman syringe filters into injection vials. The analysis was carried out using Gas Chromatography under the following conditions:—

| GC System: | 3 | |
|---|---|---|
| Column: | Phenomenex ZB-FFAP (30 m × 0.53 mm × 1 μm) | |
| Head pressure: | 6 | psi |
| Carrier gas: | Helium | |
| Split gas flow: | 15 | ml/min |
| Hydrogen gas flow: | 45 | ml/min |
| Nitrogen gas flow: | 20 | ml/min |
| Oven Program: | Initial temp: | 200° C. |
| Initial time: | 2 | min |
| Rate of ramp: | 5° | C./min |
| Final temp: | 240° | C. |
| Total Run Time: | 10 | min |
| Injector temp: | 250° | C. |
| Injection volume: | 1 | μl |
| Detector temp: | 250° | C. |

| | |
|---|---|
| Detection: | FID |

### Results

Table 3 shows the amount of lauric acid contained in each P(L)LA/LA fibre.

TABLE 3

| | Amount | Mean |
|---|---|---|
| Sample Details | (% w/w) | (% w/w) |
| PLA (3.8 IV)/Lauric acid | 0.39 | 0.38 |
| | 0.36 | |
| PLA (4.5 IV)/Lauric acid | 0.422 | 0.42 |
| | 0.412 | |

### EXAMPLE 5

### Degradation—Tensile Strength

### Method

The fibres were subjected to in vitro degradation by immersion in standard phosphate buffer solution (PBS), maintained at 37° C.

During the ten week test period samples were analysed to determine the tensile strength of the fibres using a gauge length of 40 mm and a test speed of 10 mm/min.

### Results

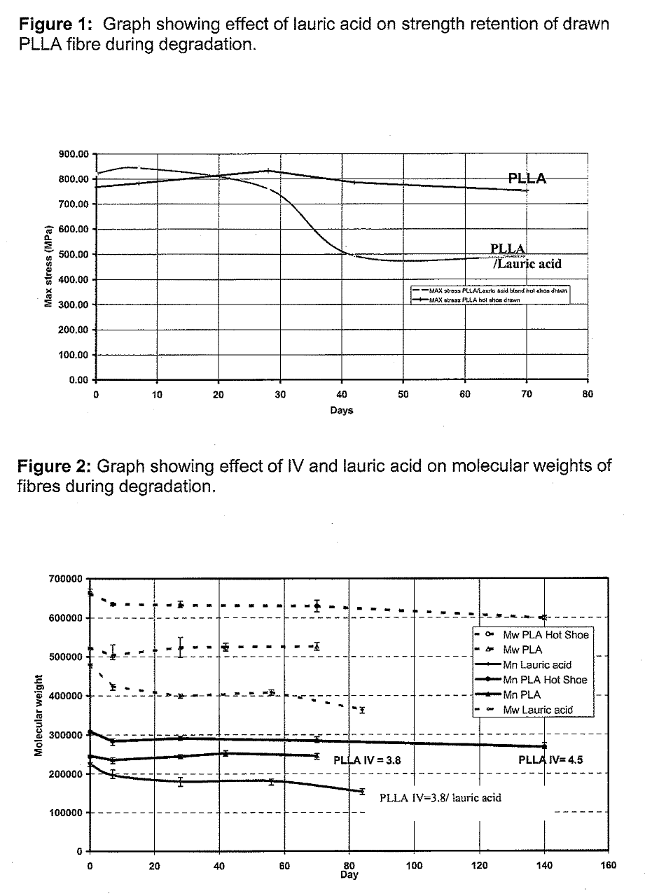

The results are reported in FIG. 1 which shows a higher tensile strength of P(L)LA/LA (0.4%) for the initial 3 weeks, thereafter declining below that of drawn P(L)LA fibres over 5 weeks. Drawn P(L)LA fibres show a steady tensile strength over the initial 10 weeks. The degradation rate of P(L)LA/LA (0.4%) could be delayed by using a precursor of LA, for example Lauric anhydride.

Samples were also analysed to determine molecular weight (Mn) of the polymer fibres during the ten week test period, the results are reported in FIG. 2.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. An oriented implantable, biodegradable device formed from a homogeneous polymer blend comprising a polylactic acid in admixture, wherein the polylactic acid is present in an amount of not more than 10% by weight of the polymer blend; and an additive which both plasticises polymer draw and is a degradation accelerant or a precursor thereof,
wherein the polymer blend comprises polymers that are in uniaxial, biaxial or triaxial orientation.

2. The oriented device of claim 1, wherein the additive is a carboxylic acid or precursor thereof.

3. The oriented device of claim 2, wherein the precursor is a carboxyl containing compound.

4. The oriented device of claim 2, wherein the precursor is an acid anhydride, an ester, or an acid precursor.

5. The oriented device of claim 2, wherein the acid is a mono or poly saturated or unsaturated acid, diacid, or precursor thereof.

6. The oriented device of claim 1, wherein the additive is selected from the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-beta-hydromuconic acid, isovaleric anhydride, hexanoic anhydride, decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride, phthalic anhydride, benzoic acid, benzoic anhydride and mixtures thereof.

7. The oriented device of claim 1, wherein the additive is lauric acid or lauric anhydride.

8. The oriented device of claim 1, wherein the polymer blend comprises not more than 2% by weight of the additive.

9. The oriented device of claim 1 wherein the polylactic acid is a homopolymer, blend, or block copolymer, and wherein the polylactic acid comprises poly(L) lactic acid, poly (D) lactic acid, or poly (DL) lactic acid.

10. The oriented device of claim 9, wherein the polylactic acid is a block copolymer comprising a polylactic acid and a polyester.

11. The oriented device of claim 1, wherein the polymeric component has a number average molecular weight (Mn) in excess of about 30,000 daltons.

12. The oriented device of claim 1, wherein the polymeric component has a number average molecular weight (Mn) of about 50,000 to about 500,000 daltons.

13. The oriented device of claim 1, wherein the polymeric component has an intrinsic viscosity (IV), of about 1 to about 10.

14. The oriented device of claim 1, wherein the polymeric component comprises at least one filler.

15. The oriented device of claim 14, wherein the filler is an osteoconductive material.

16. The oriented device of claim 1 wherein the device is in the form of fibres, drawn monoliths, spun polymer, or moulded polymer.

17. A high strength composite comprising the oriented device of claim 1, wherein the high strength composite is reinforced by component fibres, drawn monoliths, spun polymer, or moulded polymer.

18. The oriented device of claim 1, wherein the device has a tensile strength of about 150 MPa to about 2000 MPa for fibre form devices.

19. The oriented device of claim 1, wherein the device has a tensile strength of about 800 to about 2000 MPa.

20. The oriented device of claim 1, wherein the device has a tensile strength of about 800 to about 1000 MPa.

21. The oriented device of claim 1, wherein the device has a tensile strength of about 1000 to about 2000 MPa.

22. The oriented device claim 1, wherein the device has a tensile strength of about 150 to about 800 MPa.

23. A composite comprising a biodegradable polymer matrix comprising the oriented device of claim 1.

24. The composite of claim 23, wherein the polymer matrix comprises a polyester, a polylactic acid, or a blend or block copolymer thereof.

25. The composite of claim 23, wherein the matrix component is a homogeneous polymer blend comprising: (1) a polymer in admixture, wherein the polymer is present in an amount of not more than 10% by weight of the polymer blend; and (2) an additive which is a degradation accelerant or a precursor thereof, wherein the additive comprises a carboxylic acid or precursor thereof.

26. The composite of claim 23 wherein the polymer matrix comprises fillers and/or biological actives.

27. The composite of claim 23 wherein the composite is in the form of a suture anchor, soft tissue anchor, interference screw, tissue engineering scaffold, maxillo-facial plate, fracture fixation plate or rod.

28. The composite of claim 23 wherein the composite has a tensile strength of about 150 MPa to about 800 MPa.

29. The composite of claim 23 wherein the composite has a tensile strength of about 250 to about 550 MPa.

30. A process for the preparation of an oriented implantable, biodegradable device comprising the steps of:
i) preparing a polymer blend comprising a polylactic acid in admixture, wherein the polylactic acid is present in an amount of not more than 10% by weight of the polymer blend; and an additive which both plasticises polymer draw and is a degradation accelerant or a precursor thereof; and
ii) processing the polymer blend to orient at least some of the polymers whereby the oriented polymers are in uniaxial, biaxial or triaxial orientation.

31. A process for the preparation of the oriented device of claim 1 comprising the steps of: (1) preparing a polymer blend comprising a polylactic acid in admixture, wherein the polylactic acid is present in an amount of not more than 10% by weight of the polymer blend; and an additive which both plasticises polymer draw and is a degradation accelerant or a precursor thereof; and (2) processing the polymer blend to orient at least some of the polymers whereby the oriented polymers are in uniaxial, biaxial or triaxial orientation.

32. The process of claim 30, further comprising the steps of forming the polymer by casting, compression moulding, or extruding; orienting the polymer chains by aligning melt phase polymer, drawing, spinning, or moulding, wherein the polymer chains are oriented in the direction of draw, spin, axis, or direction of moulding; and cooling.

33. A process for preparing a composite comprising a biodegradable polymer matrix comprising an oriented device, the process comprising the steps of: (1) providing the oriented device of claim 1; and (2) combining the oriented device with a polymer matrix comprising a polyester a polylactic acid, or a blend or block copolymer thereof.

34. (canceled)

35. The method of claim 34, wherein the device is selected from the group consisting of plates, screws, pins, rods, anchors or scaffolds.

36. The method of claim 34, wherein the device is selected from the group consisting of suture anchors, soft tissue anchors, interference screws, tissue engineering scaffolds, maxillo-facial plates, fracture fixation plates and rods.

37. The oriented device of claim 9, wherein the polylactic acid is a block copolymer comprising polylactic acid and polyglycolic acid.

38. The oriented device of claim 37, wherein the block copolymer further comprises poly(ethylene glycol), poly(ε-caprolactone), poly(3-hydroxybutyrate), poly(p-dioxanone), poly(propylene fumarate), or poly(trimethylene carbonate).

39. The composite of claim 23 wherein the oriented device comprises fillers and/or biological actives.
